(19) United States
(12) Patent Application Publication
(10) Pub. No.: US 2011/0125228 A1
(43) Pub. Date: May 26, 2011

(54) LASER BASED MEDICAL INSTRUMENT AND METHOD HAVING INTERCHANGEABLE CARDS

(76) Inventors: Larry Lytle, Rapid City, SD (US); Alf-Kare Eide Riisnaes, Rapid City, SD (US); Kip Lytle, Rapid City, SD (US); Shawn Gab, Rapid City, SD (US)

(21) Appl. No.: 12/626,633

(22) Filed: Nov. 26, 2009

(51) Int. Cl. A61N 5/067

(52) U.S. Cl. 607/89

(57) ABSTRACT

A laser-based medical instrument and method having interchangeable cards are disclosed. A method of laser therapy includes providing one or more substantially planar laser diode(s), configured to lase at a wavelength when driven, to form a medical instrument. The method also includes focusing a resultant beam including an output beam of the one or more substantially planar laser diode(s) at the corresponding wavelength thereof on a biological medium to impart energy to the biological medium. Further, the method includes altering a mode of operation of the medical instrument when an interchangeable card of the medical instrument is removed. The mode of operation includes one or more segment(s) that include a time of pulsation of the one or more substantially planar laser diode(s) and a frequency of pulsation of the one or more substantially planar laser diode(s).

### FIELD OF TECHNOLOGY

This disclosure relates generally to laser therapy and, more particularly, to a method, an apparatus, and a system of a laser-based medical instrument and method having interchangeable cards.

### BACKGROUND

An organism (e.g., humans, animals) may require repair of constituent living cells when the living cells are diseased and/or injured. Directing a low-level laser radiation onto the portions of the biological mediums (e.g., tissue) may aid in the repair and/or reconstruction of the living cells.

A medical instrument may need to operate with modes optimized to treat different conditions (e.g., diabetes, osteoarthritis, heart conditions, etc.). The medical instrument may require separate approval of a government health agency (e.g., the Food and Drug Administration) to be employed in treating each condition. Different versions of the medical instrument having different modes may be required to treat different conditions. However, manufacturing many different versions of the medical instrument may be expensive, complicated, and/or uneconomical.

### SUMMARY

Disclosed are a method, an apparatus, and a system of a laser-based medical instrument and method having interchangeable cards.

In one aspect, a method of laser therapy includes providing one or more substantially planar laser diode(s) (e.g., configured to lase at a wavelength when driven) from a medical instrument. The method also includes directing a resultant beam including an output beam of the one or more substantially planar laser diode(s) at the corresponding wavelength thereof on a biological medium to impart energy to the biological medium. Further, the method includes altering a mode of operation of the medical instrument when an interchangeable card (for example, an identification card) of the medical instrument is replaced with another interchangeable card. The mode of operation includes one or more segment(s) that include a time of pulsation of the one or more substantially planar laser diode(s) and a frequency of pulsation of the one or more substantially planar laser diode(s). In several embodiments, the medical instrument may be a laser therapy device.

In several embodiments the treatment or therapy administered by the medical instrument to treat a biological medium may be referred to as, but is not limited to, low-level laser therapy (LLLT), laser biostimulation, laser irradiation, laser therapy, low-power laser irradiation, or low-power laser therapy. In several embodiments, the medial instrument may provide laser therapy or laser treatment to the biological medium.

In another aspect, a method of laser therapy includes coupling a first medical instrument to a second medical instrument through a hardware connector. The first medical instrument and the second medical instrument each include one or more substantially planar laser diode(s) configured to enable or disable certain laser diodes. The enablement and disablement of certain laser diodes may result in the production of radiation of different wavelengths. The method also includes altering a mode of operation of the first medical instrument and the second medical instrument when an interchangeable card of the first medical instrument and/or the second medical instrument is substituted with a new interchangeable card. The mode of operation includes one or more segment(s) that include a time of pulsation of the one or more substantially planar laser diode(s) and a frequency of pulsation of the one or more substantially planar laser diode(s).

Further, the method includes enabling communication between the first medical instrument and the second medical instrument through the hardware connector, and directing a first resultant beam including an output beam of the one or more substantially planar laser diode(s) of the first medical instrument on a first location of a biological medium to impart energy to the first location of the biological medium and/or a second resultant beam including an output beam of the one or more substantially planar laser diode(s) of the second medical instrument on a second location of the biological medium to impart energy to the second location of the biological medium.

In yet another aspect, a medical instrument includes one or more substantially planar laser diode(s), configured to lase at a wavelength when driven, and an interchangeable card to alter a mode of operation of the medical instrument when removed. The mode of operation includes one or more segment(s) that include a time of pulsation of the one or more substantially planar laser diode(s) and a frequency of pulsation of the one or more substantially planar laser diode(s). A resultant beam including an output beam of the one or more substantially planar laser diode(s) at the corresponding wavelength thereof is directed on a biological medium to impart energy to the biological medium.

Further, in another aspect, a therapeutic laser system includes a first medical instrument and a second medical instrument. The first medical instrument and the second medical instrument each include one or more substantially planar laser diode(s) configured to lase at a wavelength when driven. The therapeutic laser system also includes a hardware connector to couple the first medical instrument to the second medical instrument, and to enable communication between the first medical instrument and the second medical instrument. Further, the therapeutic laser system includes an interchangeable card of the first medical instrument and/or the second medical instrument to alter a mode of operation of the first medical instrument and the second medical instrument when the interchangeable card is substituted with a new interchangeable card.

The mode of operation includes one or more segment(s) that include a time of pulsation of the one or more substantially planar laser diode(s) and a frequency of pulsation of the one or more substantially planar laser diode(s). A first resultant beam including an output beam of the one or more substantially planar laser diode(s) of the first medical instrument and/or a second resultant beam including an output beam of the one or more substantially planar laser diode(s) of the second medical instrument are directed on a first location of a biological medium and/or a second location of the biological medium to impart energy to the first location of the biological medium and/or the second location of the biological medium.

The methods and systems disclosed herein may be implemented in any means for achieving various aspects, and may be executed in a form of a machine-readable medium embodying a set of instructions that, when executed by a machine, cause the machine to perform any of the operations disclosed herein. Other features will be apparent from the accompanying drawings and from the detailed description that follows.

### BRIEF DESCRIPTION OF THE DRAWINGS

The embodiments of this invention are illustrated by way of example and are not limited to the figures of the accompanying drawings, in which like references indicate similar elements and in which:

FIG. 1 is a process flow detailing the operations involved in a method of laser therapy, according to one or more embodiments.

FIG. 2 is a schematic view of a medical instrument, according to one or more embodiments.

FIG. 3 is an illustrative view of the mode display in the medical instrument of FIG. 2, according to one or more embodiments.

FIG. 4 is an illustrative view of a user control capability in the medical instrument, according to one or more embodiments.

FIG. 5 is a flowchart illustrating the operations involved in reprogramming the medical instrument, according to one or more embodiments.

FIG. 6 is a process flow detailing the operations involved in a method of laser therapy involving coupling of a medical instrument to another medical instrument, according to one or more embodiments.

FIG. 7 is a system view of a first medical instrument coupled to a second medical instrument, according to one or more embodiments.

FIG. 8 is a flowchart detailing the operations involved in synchronizing the first medical instrument and the second medical instrument of FIG. 7, according to one or more embodiments.

FIG. 9 is a system view of a probe device, according to one or more embodiments.

FIG. 10 is a system view of the medical instrument of FIG. 4 operating in unison with the probe device of FIG. 9, according to one or more embodiments.

Other features of the present embodiments will be apparent from the accompanying drawings and from the detailed description that follows.

### DETAILED DESCRIPTION

Example embodiments, as described below, may be used to provide a method, a system, and an apparatus of a laser-based medical instrument and method having interchangeable cards. Although the present embodiments have been described with reference to specific example embodiments, it will be evident that various modifications and changes may be made to these embodiments without departing from the broader spirit and scope of the various embodiments.

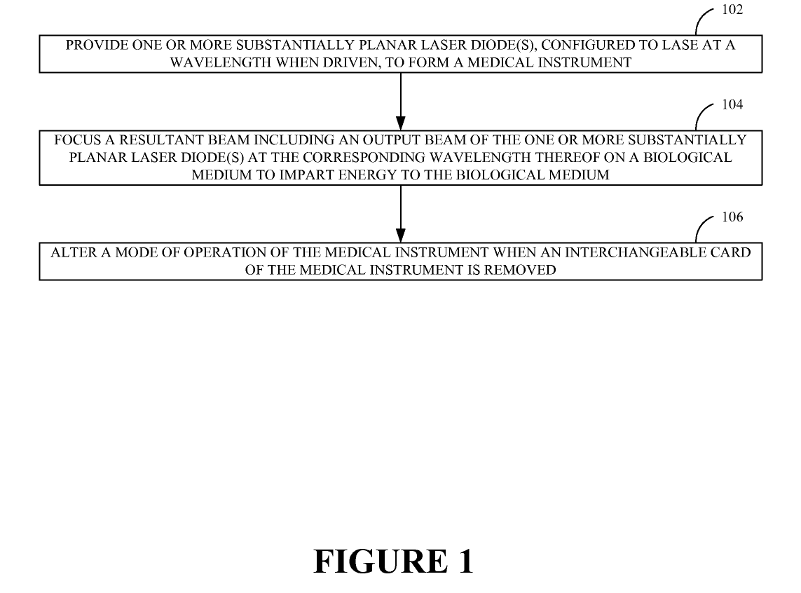

FIG. 1 is a process flow detailing the operations involved in a method of laser therapy, according to one or more embodiments. In operation 102, one or more substantially planar laser diode(s), each configured to lase at a wavelength when driven, may be provided to form a medical instrument. In one or more embodiments, a number of substantially planar laser diodes may be arranged in a pre-determined configuration to form a substantially planar laser diode array. In one or more embodiments, the substantial planarity, along with a symmetrical pre-determined configuration, may provide for a symmetrical combination of the output beams from the number of substantially planar laser diodes to form a resultant beam.

In one or more embodiments, soliton waves may be generated from the one or more substantially planar laser diode(s). In one or more embodiments, end mirrors of the one or more substantially planar laser diode(s) may be replaced with anti-reflection coatings, and when the one or more substantially planar laser diode(s) are driven, the optical field evolution in the laser diode(s) may be modeled by using two coupled differential equations (example Equations 1 and 2) as:

$$\begin{matrix}\frac{\partial ψ}{\partial z} = \frac{i {\partial }^{2} ψ}{2 \partial {x}^{2}} + ( - i h N + ( N - 1 ) - α ) ψ , and & ( 1 ) \\ D \frac{{\partial }^{z} N}{\partial {x}^{z}} = - π + N + B {N}^{z} + C {N}^{z} + ( N - 1 ) {\vert ψ \vert}^{z} , & ( 2 )\end{matrix}$$

where ψ may be the optical field solution, i=\sqrt{−1}, x and z the spatial coordinates, h the Henry factor, α the internal loss, N the normalized carrier density

$$( N = \frac{{N}^{′}}{{N}_{t τ}^{′}} , {N}^{′}$$

being the carrier density, and N [symbol] ′ being the transparency carrier density), D the carrier diffusion coefficient, π the current pumping coefficient, B the spontaneous recombination coefficient, and C the Auger recombination rate. Here, a linear dependence of the induced refractive index and gain on the carrier density N′ may be assumed.

In one or more embodiments, neglecting carrier diffusion in the z direction, and assuming small diffusion, B=0, and C=0, a generalized complex Ginzburg-Landau equation may be obtained from Equations 1 and 2 as example Equation 3:

$$\frac{\partial ψ}{\partial z} = i ( \frac{1}{2} - i β ) \frac{{\partial }^{z} ψ}{\partial {x}^{z}} + ( \frac{π - 1}{1 + {\vert ψ \vert}^{z}} ( - i h + 1 ) - i h ) ψ - α ψ , \qquad ( 3 )$$

where β may account for the transverse carrier diffusion.

In one or more embodiments, soliton wave solutions of the form ψ(x)^{tλ} [symbol] may be numerically obtained. In one or more embodiments, depending on the arrangement of the number of substantially planar laser diodes, constructive interference of the outputs of the number of substantially planar laser diodes may lead to a resultant soliton wave of high amplitude. In one or more embodiments, the resultant soliton wave output may have an amplitude several times higher than a non-soliton wave resultant beam.

In operation 104, the resultant beam may be focused on a biological medium to impart energy to the biological medium (e.g., humans, animals). In one or more embodiments, the resultant beam may be directed on a portion of the human body to treat conditions such as osteoarthritis. In one or more embodiments, in operation 106, a mode of operation of the medical instrument may be altered upon removal of an interchangeable card of the medical instrument. In one or more embodiments, the interchangeable card may be therapeutic condition specific (e.g., osteoarthritis, diabetes, veterinary condition), and the insertion of a new interchangeable card into the medical instrument may result in the medical instrument operating solely in modes of operation specific to the therapeutic condition. In other words, access to mode information is restricted to modes of operation specific to the therapeutic condition.

In one or more embodiments, altering the mode of operation of the medical instrument upon removal of the interchangeable card, as in operation 106, may involve substituting the interchangeable card with another interchangeable card. In one or more embodiments, one interchangeable card may be specific to one therapeutic condition (e.g., osteoarthritis), and the other interchangeable card may be specific to another therapeutic condition (e.g., diabetes).

In one or more embodiments, a mode of operation may include one or more segments, where a segment includes a time of pulsation of the one or more substantially planar laser diode(s) and a frequency of pulsation of the one or more substantially planar laser diode(s). For example, one segment may include pulsing a laser diode at 50 Hz for 20 seconds, and another segment may include pulsing a laser diode at 10 Hz for 30 seconds. In one embodiment, a mode may consist of up to 250 different segments.

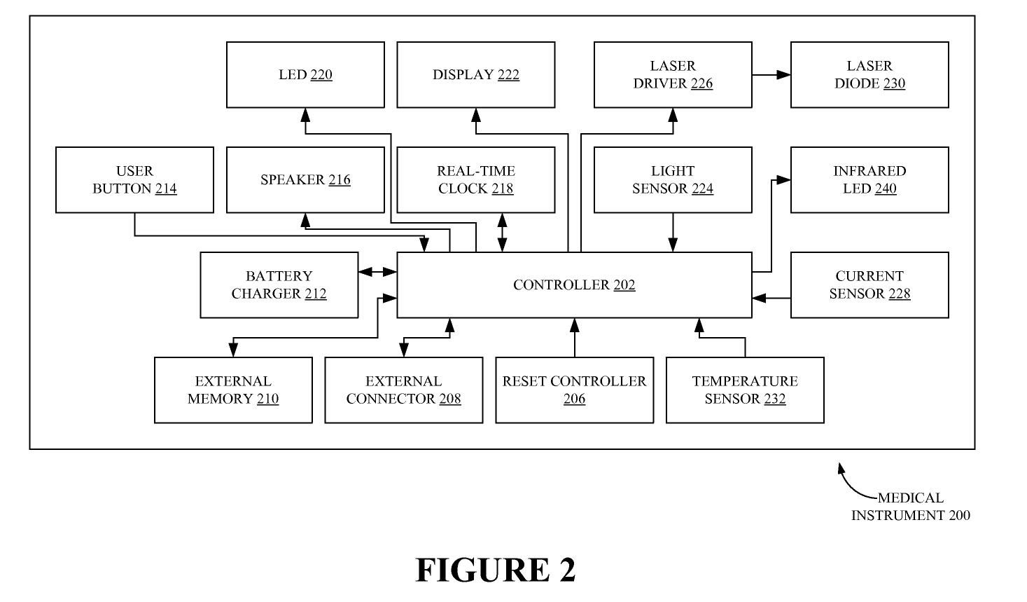

FIG. 2 is a schematic view of a medical instrument 200, according to one or more embodiments. In one or more embodiments, the medical instrument 200 may include a controller 202 to control operations fundamental to the working of the medical instrument 200. In one or more embodiments, the controller 202 may include a permanent memory (e.g., flash memory) to store firmware associated with controlling the medical instrument 200. In one or more embodiments, modes of operation may be internally set in the firmware. In one or more embodiments, the controller 202 is interfaced with a battery charger 212 to charge a battery (e.g., internal battery) of the medical instrument 200. In one or more embodiments, the battery charging capability may be provided through an external connector 208 that may serve purposes not limited to battery charging.

In one or more embodiments, the external connector 208 may be a multi-pin and multi-use external connector that may also be used to program the internal controller of the medical instrument 200 (e.g., controller 202), to calibrate constituent laser diodes 230, to couple other external compatible devices (e.g. another medical instrument 200, a probe version of the medical instrument 200, a computer device, a personal digital assistant (PDA)), and/or to perform diagnostics of the medical instrument 200.

In one embodiment, the medical instrument 200 may be powered by a lithium-ion rechargeable battery placed in an inside thereof. Here, the battery charger may plug into the medical instrument 200 through the external connector 208, and may closely monitor charge current as well as maximum allowed voltage. In one or more embodiments, the battery may be supplied with a safety circuitry to prevent over-charging/over-discharging of the battery. In one or more embodiments, constituent components of the medical instrument 200 may be powered during charging of the battery, but user interaction with the medical instrument 200 may not be possible.

In one or more embodiments, the controller 202 may be interfaced with an external memory 210 to enable the medical instrument 200 to record data indicating a diagnostic requirement of the medical instrument 200. In one or more embodiments, the recorded data may be useful in enabling servicing of the medical instrument 200. For example, corrective diagnostics may be performed on the medical instrument 200 by service personnel following a return of the medical instrument 200 by a user. In one or more embodiments, the external memory 210 may be a non-volatile memory such as an Electrically Erasable Programmable Read-Only Memory (EEPROM).

In one or more embodiments, the medical instrument 200 may be provided with a user button 214 (shown in FIG. 2 as turning on the controller 202) to simplify operations thereof. In one embodiment, the user button 214 may serve as both the power ON/OFF button and the mode selection button.

In one or more embodiments, the medical instrument 200 may be provided with a speaker 216 (shown in FIG. 2 as being controlled by the controller 202) to generate audible alerts as well as indicate the pressing of the user button 214. In one or more embodiments, the audible alerts may indicate one or more of an operational status of the medical instrument 200, a beginning of a mode of operation, a beginning of a segment, an end of a mode of operation, and an end of the segment. In one or embodiments, all audible alerts may be muted by the user during use of the medical instrument 200.

In one or more embodiments, to enhance serviceability of the medical instrument 200, a real-time clock 218 (shown in FIG. 2 as being interfaced with the controller 202) may be implemented in the medical instrument 200. In one or more embodiments, data recorded in the external memory 210 may always be tagged with a current date and time at the time of recording. In one or more embodiments, this may enable a history of use of the medical instrument 200 to be tracked. For example, when a medical instrument 200 is returned to the service personnel, the service personnel may be better equipped to understand problems associated with the functioning of the medical instrument 200.

In one or more embodiments, the medical instrument 200 may be equipped with one or more Light Emitting Diodes 220 (LEDs) and a display 222 (e.g., seven segment display) that serve as user indicators. In FIG. 2, the LEDs 220 and the display 222 are shown as being controlled by the controller 202. In one embodiment, the operational state of the medical instrument 200 may be indicated with an LED emitting green light that may turn red during a power down. Here, another LED may be provided to indicate battery state and battery charging. For example, if the light emitted by this LED turns yellow during normal operation, it may be indicative of a low power level of the battery. The battery may then need to be charged. The LED may emit red light in a blinking state until charging may be complete, following which the LED may continue to emit green light. In one or more embodiments, the display 222 may indicate modes that are loaded onto the medical instrument 200, and, in one embodiment, the modes may be indicated on the display as 0-9. Here, the user may select a mode using the mode selection feature of the user button 214.

In one or more embodiments, one of the purposes of the controller 202 may be to control the laser diodes 230 through laser drivers 226 thereof. In one or more embodiments, the controller 202 may control the power level of the laser diodes 230, and also the flashing of the laser diodes 230. In addition, in one or more embodiments, the controller 202 may monitor a light sensor 224 that measures the ambient light outside the medical instrument 200. This measurement may be used to control the light intensity of the user indicator LEDs 220.

In one or more embodiments, the controller 202 may have the ability to sense the operating current of each laser diode 230 (see current sensor 228 in FIG. 2), which may be used to deactivate laser diodes 230 that may have failed. In one or more embodiments, this may ensure safety of operation of the medical instrument 200. In one or more embodiments, current may also be sensed during calibration of the medical instrument 200 to ensure proper operation of the laser diodes 230. In one or more embodiments, a power management circuitry of the laser diodes 230 may be controlled by the controller 202. In one or more embodiments, infrared light may also be emitted from the infrared LEDs 240.

In one or more embodiments, the medical instrument 200 may also include a number of infrared LEDs 240 (shown as being controlled in FIG. 2 by the controller 202) to emit infrared light during a duration of a mode of operation. In one or more embodiments, the infrared LEDs 240 may operate in conjunction with one or more of the visible LEDs 220.

In one or more embodiments, the controller 202 may monitor a temperature sensor 232 to obtain accurate values of the temperatures of the laser diodes 230. In one or more embodiments, variations of temperature of the laser diodes 230 may also be tracked.

In one or more embodiments, the medical instrument 200 may include a reset controller 206 to monitor a reset button. For example, when a user depresses the reset button and holds the reset button for, say, 5 seconds, the reset controller 206 may send a reset signal to the controller 202 to reset the medical instrument 200. Here, 5 seconds is the threshold time period, and if a user presses the reset button for a time period exceeding the threshold time period, the medical instrument 200 may be reset.

In one or more embodiments, when the medical instrument 200 is turned ON and is in an idle state, an LED 220 indicating power may emit green light. In one or more embodiments, a shut off timer may be started internally to turn the medical instrument 200 off in case of inactivity (e.g., no further pressing of buttons) for a time period exceeding another threshold time period.

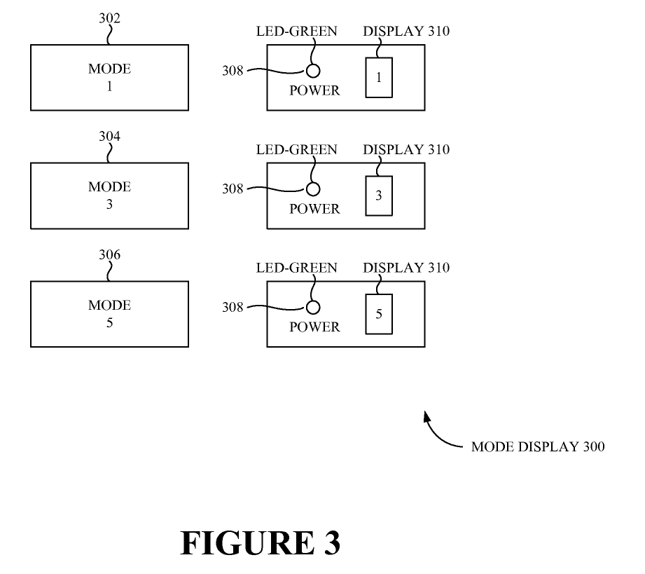

In one or more embodiments, the medical instrument 200 may be pre-programmed (e.g., by the manufacturer) with several operational modes. In one or more embodiments, the modes may be pre-programmed with the duration of treatment for a therapeutic condition, and the specific frequencies the medical instrument 200 may be operating at. FIG. 3 illustrates the mode display 300 in the medical instrument 200, according to one or more embodiments. Here, the display 310 is analogous to the display 222 in FIG. 2. In one or more embodiments, when one mode 302-306 is chosen, say mode 1 (as shown in the display 310 in FIG. 3), the power LED 308 (one of the LEDs 220) may emit green light as long as the modes 302, 304, and 306 (1, 3, and 5 illustrated in FIG. 3) are operational.

In one or more embodiments, the display 310 may slowly flash the selected mode 302-306 during the therapy session. In one or more embodiments, the end of the session may be audibly alerted, following which all laser diodes 230 and the LEDs (220, 240) may be turned off. In one or more embodiments, as discussed above, the battery LED 220 may emit yellow light to indicate the low power level state of the internal battery, whereupon the user may recharge the battery by inserting the battery charger 212. In one or more embodiments, the battery charging state may also be indicated through an LED 220.

In one or more embodiments, data indicating diagnostic information of the medical instrument 200 may include one or more of a number of times the medical instrument 200 is turned ON/OFF, a number of times the medical instrument 200 is recharged, a temperature variation of the medical instrument 200, a number of times a mode of operation is used, a data indicating a lifespan of the laser diode 230, and a data from the real-time clock 218 in the medical instrument 200 indicating a history of use.

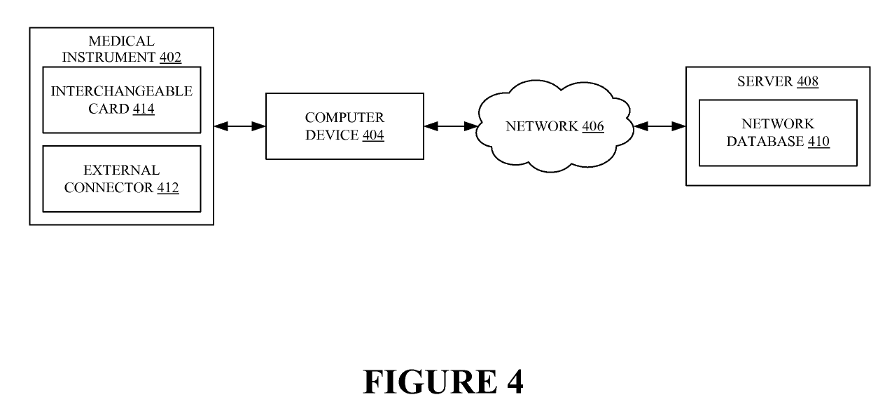

In one or more embodiments, as discussed above, coupling of an external computer device to the medical instrument 200 may be possible through the external connector 208. FIG. 4 illustrates a user control capability in the medical instrument 402 (analogous to the medical instrument 200), according to one or more embodiments. In one or more embodiments, a computer device 404 may be coupled to the medical instrument 402 through the external connector 412 (analogous to external connector 208). For example, the medical instrument 402 may plug into the computer device 404 (e.g., personal computer) through a Universal Serial Bus (USB) port of the computer device 404, and the medical instrument 402 may be identified by the computer device 404 as a removable hard drive.

In one or more embodiments, the aforementioned coupling of the computer device 404 to the medical instrument 402 may enable access to a network database 410 residing on a server 408 through a network 406 (e.g., Internet). In one or more embodiments, this may be accomplished by a website linking to the network database 410 opening up and/or a user of the medical instrument 402 opening the website. In one or more embodiments, the medical instrument 402 may plug into the computer device 404 through the external connector 412 using a binary serial connection, Recommended Standard 232 (RS-232).

In one or more embodiments, the user may enter a serial number (S/N), and the computer device 404 may detect the medical instrument 402 to then locate a match in the network database 410. In one or more embodiments, the computer device 404 and/or the user may then be aware of the modes pre-programmed into the medical instrument 402. In one or more embodiments, the S/N may be transmitted automatically to start the website upon coupling of the medical instrument 402 to the computer device 404. In one or more embodiments, modes of operation not limited to the pre-programmed modes available in the medical instrument 402 may be accessed by way of the network database 410. In one or more embodiments, access to the modes of operation through the website may be further secured by provision of a user ID and a password to log into the website.

In one or more embodiments, the user may be ready to control the modes of operation in the medical instrument 402. In one or more embodiments, user control capability may be accomplished by way of enabling the user to program the medical instrument 402. In one or more embodiments, the user may program modes of operation including segments of each mode. In one or more embodiments, when the user may be ready to program the medical instrument 402, files associated with modes of operation may be assembled in an organized manner to enable the user to choose desired modes. In one or more embodiments, a file chosen by the user may be encrypted with the S/N entered by the user, as discussed above. In one or more embodiments, the S/N (or encryption key) may also be automatically obtained to encrypt the file chosen by the user.

In one or more embodiments, the file chosen by the user may be transferred to the external memory 210 of the controller 202 in an encrypted format. In one embodiment, the medical instrument 402 may be regarded by the computer device 404 as a USB drive. In one or more embodiments, upon receipt of the file chosen by the user, the medical instrument 402 may decrypt the file to go through a reprogramming process to let the new modes of operation associated with the file take over. In one or more embodiments, the user may turn off the medical instrument 402 after the chosen file is transmitted and, upon powering the device the next time, the file from the external memory 210 of the medical instrument 402 may be located and decrypted using the S/N prior to a self reprogramming process.

In one or more embodiments, user uploading of files to the network database 410 may also be enabled. In one embodiment, diagnostic information may be provided by the medical instrument 402 in an encrypted format, and the diagnostic information may be uploaded to the network database 410. In one or more embodiments, modes of operation may be shared through the website between one medical instrument having a first encryption key and another medical instrument having a second encryption key. In one or more embodiments, the sharing of modes between medical instruments may be restricted to the website. In one or more embodiments, users may submit feedback about the medical instrument 402 at the website. In one or more embodiments, snippets of user feedback may be used by a manufacturer of the medical instrument 402 for marketing purposes.

In one or more embodiments, an interchangeable card 414 may be provided in the medical instrument 402, as shown in FIG. 4. In one or more embodiments, the insertion of the interchangeable card 414 into the medical instrument 402 may complete a circuit path between the interchangeable card 414 and the controller 202. In one or more embodiments, the interchangeable card 414 may communicate with the controller 202 to control modes of operation to be stored in the external memory 210 through instructions programmed into the firmware stored in a permanent memory of the controller 202. In one or more embodiments, the permanent memory of the controller 202 may be cleared after the modes of operation are stored in the external memory 210.

In one or more embodiments, the interchangeable card 414 may be a Printed Circuit Board (PCB) having traces that are employed in the completion of the circuit path upon insertion of the interchangeable card 414 into the medical instrument 402. In one or more embodiments, the interchangeable card 414 may serve as a therapeutic condition specific label that may be supplied with the medical instrument 402 during purchase. In one or more embodiments, the interchangeable card 414 may restrict the utility of the medical instrument 402 to solving problems associated with a specific therapeutic condition (e.g., osteoarthritis). In this case, the medical instrument 402 may be sold as an osteoarthritis unit. In one or more embodiments, the interchangeable card 414 may have an internal active chip to provide the identification features.

In one or more embodiments, the insertion of the interchangeable card 414 into the medical instrument 402 may restrict downloading of modes of operation associated with other therapeutic conditions from the network database 410 even after coupling of a computer device 404 to the medical instrument 402. In one or more embodiments, downloading modes of operation associated with another therapeutic condition (e.g., diabetes) may only be possible upon substitution of the interchangeable card 414 with another interchangeable card 414 specific to the new therapeutic condition (e.g., diabetes). In one or more embodiments, no matter what may be programmed in the firmware, as soon as a new therapeutic condition specific interchangeable card 414 is inserted into the medical instrument 402, the reprogramming of firmware may render the medical instrument 402 specific to solving problems associated with the new therapeutic condition.

Therefore, in one or more embodiments, the mode of operation of the medical instrument 402 may be altered upon removal of the interchangeable card 414 and, also, upon insertion of a new interchangeable card 414.

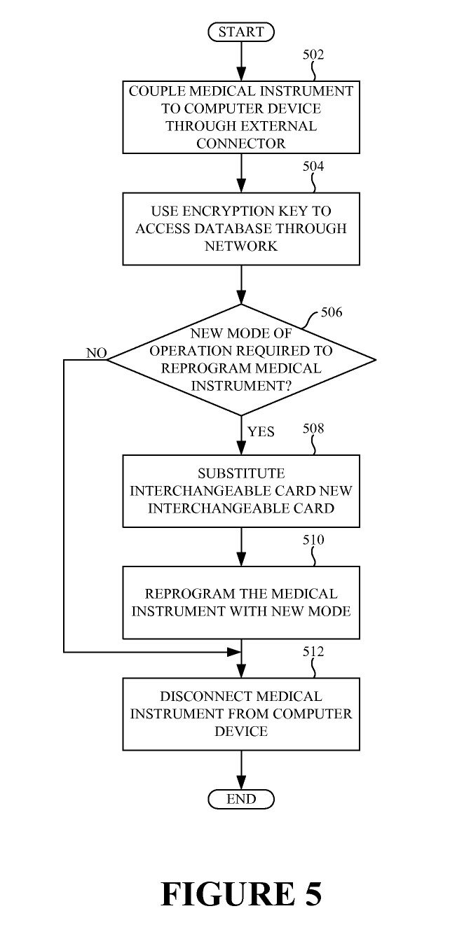

FIG. 5 is a flowchart illustrating the operations involved in reprogramming the medical instrument 402, according to one or more embodiments. In operation 502, the medical instrument 402 may be coupled to the computer device 404 through the external connector 412. In operation 504, the encryption key may be used to access the network database 410 through the network 406. In operation 506, the requirement of a new mode to reprogram the medical instrument 402 with may be checked for. If yes, the interchangeable card 414 may be substituted with a new interchangeable card 414 in operation 508. The medical instrument 402 may then be reprogrammed with a new mode of operation specific to a therapeutic condition associated with the new interchangeable card 414 in operation 510. The medical instrument 402 may then be disconnected from the computer device 404, as in operation 512. If there is no requirement to reprogram the medical instrument 402 in operation 506, the medical instrument 402 may, again, be disconnected from the computer device 404, as in operation 512.

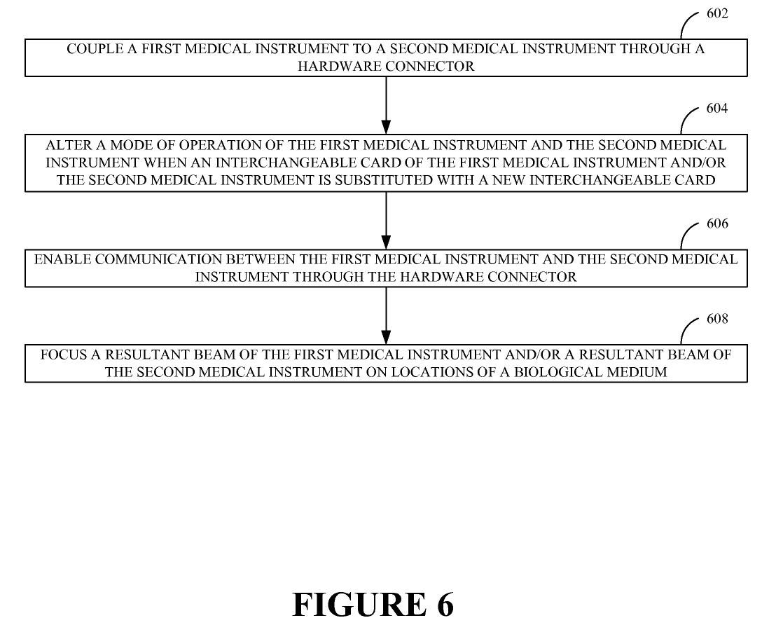

FIG. 6 is a process flow detailing the operations involved in a method of laser therapy involving coupling of a medical instrument 402 to another medical instrument 402, according to one or more embodiments. In operation 602, a first medical instrument may be coupled to a second medical instrument through a hardware connector. In one or more embodiment, the first medical instrument and the second medical instrument may each comprise one or more substantially planar laser diode(s), each configured to lase at a wavelength when driven. In one or more embodiments, the first medical instrument and the second medical instrument may each comprise an array of substantially planar laser diodes. In operation 604, a mode of operation of the first medical instrument and the second medical instrument may be altered when an interchangeable card of the first medical instrument and/or the second medical instrument may be substituted with a new interchangeable card.

In one or more embodiments, the mode of operation may include one or more segment(s) including a time of pulsation of the one or more substantially planar laser diode(s) and a frequency of pulsation of the one or more substantially planar laser diode(s), as discussed above. In one or more embodiments, the substitution of the interchangeable card with the new interchangeable card may enable a new mode of operation specific to a therapeutic condition associated with the new interchangeable card, and disable the mode of operation specific to a therapeutic condition associated with the interchangeable card. The interchangeable card is analogous to the interchangeable card described in FIG. 4, and the relevant discussion with regard to FIG. 4 will suffice for the description of the interchangeable card here.

In operation 606, communication between the first medical instrument and the second medical instrument may be enabled through the hardware connector. In one or more embodiments, the enabling of the communication between the first medical instrument and the second medical instrument may provide compatibility to render the first medical instrument compatible with the second medical instrument and/or synchronize a mode of operation between the first medical instrument and the second medical instrument. In one or more embodiments, enabling communication between the first medical instrument and the second medical instrument may involve utilizing a communication protocol (e.g., Inter-Integrated Circuit) to facilitate the communication process.

In one or more embodiments, synchronizing the mode of operation between the first medical instrument and the second medical instrument may include communicating to the hardware connector the mode of operation specific to a therapeutic condition associated with the new interchangeable card to be executed. In one or more embodiments, the first medical instrument and the second medical instrument may then be prepared to receive the mode of operation through the hardware connector. In one or more embodiments, the mode of operation may then be sent bidirectionally to both the first medical instrument and the second medical instrument at the same time. In one or more embodiments, this may involve executing a same set of instructions on both the first medical instrument and the second medical instrument. In one or more embodiments, the communication protocol facilitating the communication process may include executing a cycle redundancy check (CRC) to ensure integrity of the communication process. In one or more embodiments, the hardware connector may include a flash programmable microcontroller including a flash memory to control the communication process.

In one or more embodiments, the first medical instrument may be used to power the second medical instrument upon mutual coupling through the hardware connector. In one or more embodiments, the first medical instrument may power an older version of the second medical instrument, thereby providing backward compatibility. In one or more embodiments, the first medical instrument may have an operating voltage different from that of the second medical instrument. In one or more embodiments, compatibility between the devices may be provided by way of a step-up/step-down transformer circuit to scale down/scale up a voltage level.

In one or more embodiments, the one or more substantially planar laser diode(s) of the first medical instrument may be turned off prior to powering the second medical instrument. In one or more embodiments, the first medical instrument and the second medical instrument may be a similar kind of device. In one or more embodiments, the first medical instrument may be a source device and the second medical instrument may be a device on which the mode of operation is to be executed. In one or more embodiments, the laser diode(s) of both the first medical instrument and the second medical instrument may be employed during the execution of modes of operation on both the first medical instrument and the second medical instrument. In one or more embodiments, the first medical instrument may be the source device and the second medical instrument may be a probe device.

In operation 608, a first resultant beam and/or a second resultant beam including an output beam of the one or more substantially planar laser diode(s) of the first medical instrument and/or the second medical instrument may be focused on a location and/or another location of a biological medium (e.g., humans, animals) to impart energy to the corresponding location(s) of the biological medium. For example, the first resultant beam may be focused onto an elbow of a human, and the second resultant beam may be focused onto a knee of the human. In another example, the two resultant beams may be focused on both the temples of the human, with the first medical instrument being on the right side and the second medical instrument being on the left side. In this case, the modes of operation at both temples may be synchronized, as discussed above.

In one or more embodiments, a resultant soliton wave may be generated from a diode array arrangement of the laser diode(s) of the first medical instrument and/or the second medical instrument, as discussed with regard to FIG. 1. In one or more embodiments, translating the first medical instrument and/or the second medical instrument away from the location of the biological medium may reduce the dosage level (i.e., radiation level). In one or more embodiments, this may increase the utility of the medical instrument as the same medical instrument may be used on organisms (e.g., human babies, animal babies) requiring a lower dosage level without the need for another medical instrument providing the lower dosage level.

In one or more embodiments, one or more segment(s) in the mode of operation of the first medical instrument or the second medical instrument may be time delayed when compared to one or more segment(s) of the mode of operation in the corresponding other second medical instrument or the first medical instrument. In one or more embodiments, the time of pulsation and/or the frequency of pulsation of the one or more substantially planar laser diode(s) of the first medical instrument or the one or more substantially planar laser diode(s) of the second medical instrument may be changed when compared to the time of pulsation and/or the frequency of pulsation of the one or more substantially planar laser diode(s) of the other second medical instrument or the one or more substantially planar laser diode(s) of the first medical instrument.

In one or more embodiments, one or more segments being executed in the first medical instrument may be different from the concurrent one or more segments being executed in the second medical instrument. For example, segments of the individual medical instruments may be mutually delayed by half a time period, by a quarter time period, or by any time period. In another example, segments of the individual medical instruments may not be mutually time delayed. In one or more embodiments, the beginning and ending of the mode of operation may be synchronized. In one or more embodiments, audible alerts may be generated during the beginning of the mode of operation, the beginning of a segment, the end of the mode of operation, and/or the end of the segment.

In one or more embodiments, the end of the mode of operation may be notified by the first medical instrument or the second medical instrument to the hardware connector.

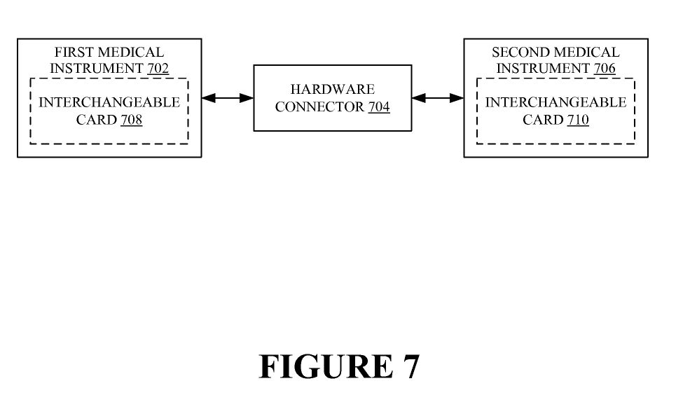

FIG. 7 is a system view of a first medical instrument 702 coupled to a second medical instrument 706, according to one or more embodiments. The first medical instrument 702 and the second medical instrument 706 are analogous to the medical instrument 402 in FIG. 4. As discussed above, in one or more embodiments, the first medical instrument 702 is coupled to the second medical instrument 706 through the hardware connector 704 (again analogous to the hardware connector discussed with regard to FIG. 6). In one or more embodiments, the presence of an interchangeable card 708/710 (analogous to the interchangeable card 414 in FIG. 4) in the first medical instrument 702 and/or the second medical instrument 704 may restrict utility of the first medical instrument 702 and/or the second medical instrument 704 to a specific therapeutic condition with associated mode(s) of operation.

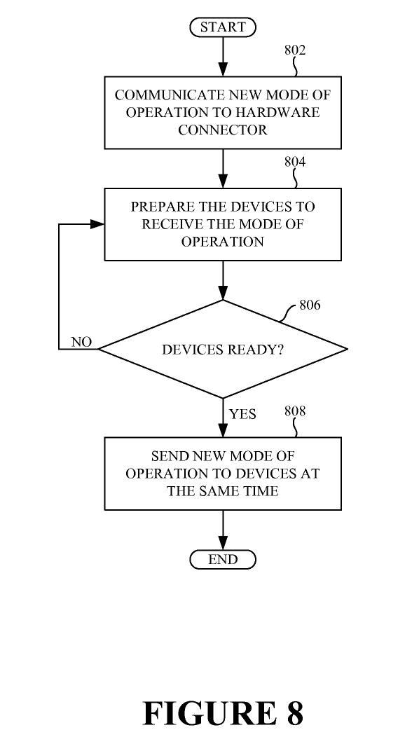

FIG. 8 is a flowchart detailing the operations involved in synchronizing the first medical instrument 702 and the second medical instrument 706, according to one or more embodiments. In operation 802, the new mode of operation specific to the new interchangeable card 708/710 may be communicated to the hardware connector 704. In operation 804, the first medical instrument 702 and the second medical instrument 706 may be prepared to receive the new mode of operation through the hardware connector 704. In operation 806, a check may be made as to whether the first medical instrument 702 and the second medical instrument 706 are ready to receive the new mode of operation. If yes, the new mode of operation may be sent at a same time to both the first medical instrument 702 and the second medical instrument 706, as in operation 808. If no, the first medical instrument 702 and second medical instrument 706 may be further prepared to receive the new mode of operation through the hardware connector 704 until they are ready to receive the new mode of operation.

As discussed above, the first medical instrument 702 and the second medical instrument 706 may be similar devices operating in unison. In one or more embodiments, where there is a requirement of directed, high-power dosage in a narrow region of a biological medium, the second medical instrument 706, for example, may be a probe device.

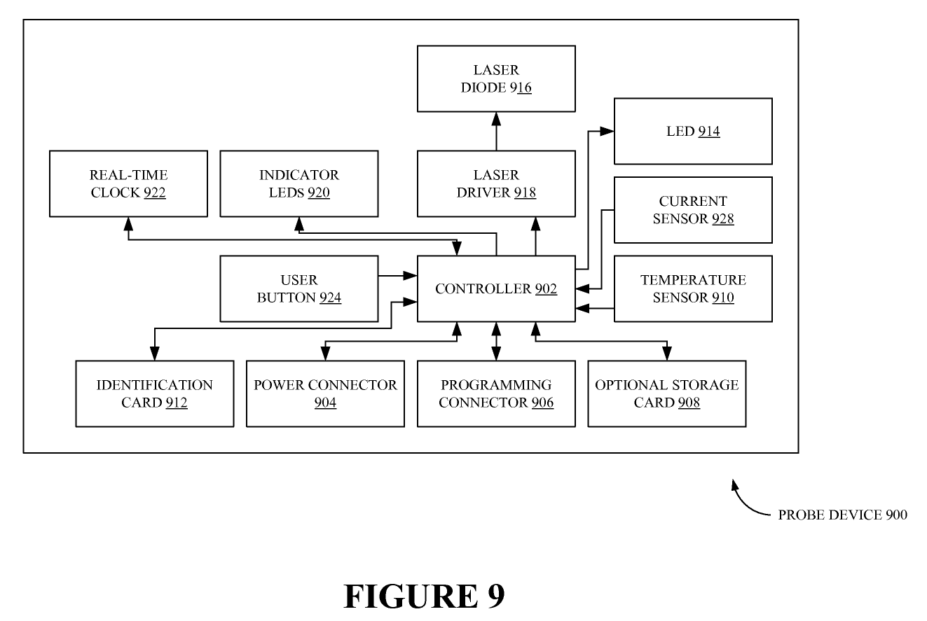

FIG. 9 is a system view of a probe device 900, according to one or more embodiments. In one or more embodiments, the probe device 900 may include a controller 902 to control all components of the probe device 900. In one or more embodiments, an operating program of the controller 902 may be user-upgraded using an optional storage card 908. In one or more embodiments, the optional storage card 908 may be a flash card from which different programs may be read.

In one or more embodiments, the probe device 900 includes a power connector 904 through which a battery of the probe device 900 may be charged. In one or more embodiments, a medical instrument 402 may be used to power the probe device 900 through the power connector 904. In one or more embodiments, the probe device 900 may include an identification card 912. The identification card 912 may include information regarding types of treatment modes to be activated. The information on the identification card 912 may be read by controller 902.

In one or more embodiments, the probe device 900 may include a programming connector 906 through which a programming/calibration interface may be provided. In one or more embodiments, the probe device 900 may be calibrated by a manufacturer and/or serviced by service personnel through the programming connector 906. In one or more embodiments, a computer device 404 may be coupled to the probe device 900 through the programming connector 906. In one or more embodiments, the programming connector 906 may not be available to a user but only available to the manufacturer and/or service personnel.

In one or more embodiments, an integrated laser driver 918 may control a laser diode 916 of the probe device 900. In one or more embodiments, an operating current of the laser diode 916 and/or a light output of the laser diode 916 may be monitored to maintain a constant output of the laser diode 916. In one or more embodiments, the laser diode 916 may be calibrated during the manufacturing process and/or the laser driver 918 may be configured to handle a range of laser diodes.

In one or more embodiments, LEDs (914, 920) may be provided to indicate an operational state of the probe device 900. A light from an LED 914 may also indicate that the optional storage card 908 is properly inserted and recognized. In another example, a number of LEDs 920 may indicate modes selected and/or progress during boot-up. In one or more embodiments, a separate LED 914 may indicate activity of the laser diode 916.

In one or more embodiments, in order for corrective diagnostics to be performed by service personnel and/or operating statistics to be obtained by the manufacturer, a real-time clock 922 may be provided in the probe device 900. In one or more embodiments, the real-time clock 922 may be programmed during manufacturing. In one embodiment, power to the real-time clock 922 may be supplied by a lithium-ion battery of the probe device 900. In another embodiment, power to the real-time clock 922 may be supplied by a coin cell battery of the probe device 900.

In one or more embodiments, the controller 902 may monitor the current of the laser diode 916 during operation of the laser diode 916 through a current sensor 928. In one embodiment, the current data may be used in the calibration of the probe device 900.

In one or more embodiments, a temperature sensor 910 may be provided in the probe device 900 to monitor a temperature of the laser diode 916 in order to ensure safety of operation of the probe device 900.

In one or more embodiments, when the probe device 900 is powered up, green light may be emitted from an LED 920. In one embodiment, when the optional storage card 908 is not present, the green LED 920 may start to blink to indicate the need to insert the optional storage card 908. In one or more embodiments, upon insertion of the identification card 912 and checking for updates residing in the identification card 912, modes of operation may be downloaded into the probe device 900. In one or more embodiments, modes of operation present on the identification card 912 may be loaded.

In one or more embodiments, user selection of modes of operation may be accomplished through a user button 924. In one or more embodiments, the probe device 900 may be turned on by a user holding the user button 924 for a time period exceeding a threshold time period of, say, 5 seconds. In one or more embodiments, a warning LED 914 may be provided to indicate a state where a laser diode 916 operating at a wavelength outside the visible spectrum may be used. In one or more embodiments, the probe device 900 may also be turned off by a user depressing the user button 924 for a time period exceeding another threshold time period.

In one or more embodiments, if at any point the identification card 912 is removed, the laser diode 916 may be turned off, and the probe device 900 may return to a boot-up state thereof.

In one or more embodiments, one or more substantially planar laser diode(s) of medical instrument 402 may lase at a wavelength of approximately ˜650 nm, ˜780 nm or ˜808 nm. In one or more embodiments, the medical instrument 402 may operate at a power level of approximately ˜42 mW. In one or more embodiments, the probe device 900 may lase at a wavelength of approximately ˜660 nm or ˜808 nm. In one or more embodiments, the probe device 900 may operate at a power level of approximately ˜50 mW or ˜500 mW. In one or more embodiments, the high power level of the probe device 900 may provide for deeper penetration into a biological medium (e.g., tissue in a human body).

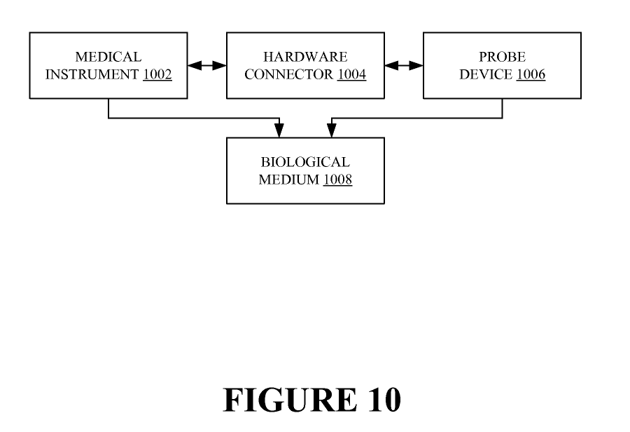

FIG. 10 is a system view of a medical instrument 1002 (analogous to the medical instrument 402 in FIG. 4) operating in unison with a probe device 1006 (analogous to probe device 900 in FIG. 9), according to one or more embodiments. In one or more embodiments, the medical instrument 1002 may power the probe device 1006 through the hardware connector 1004 (analogous to the hardware connector 704). In one or more embodiments, resultant beams of both the medical instrument 1002 and the probe device 1006 may be focused onto locations of the biological medium 1008 (e.g., humans, animals).

In one or more embodiments, the medical instrument 1002 may include a housing appropriately sized and shaped for convenient portability of the medical instrument 1002. In one or more embodiments, one or more substantially planar laser diode(s) of the medical instrument 1002 may be mounted on a recessed portion of the housing of the medical instrument 1002. In one or more embodiments, both the medical instrument 1002 and the probe device 1006 may be portable devices (e.g., hand-held devices).

Although the present embodiments have been described, with reference to specific example embodiments, it will be evident that various modifications and changes may be made to these embodiments without departing from the broader spirit and scope of the various embodiments. For example, the various devices and modules described herein may be enabled and operated using hardware circuitry (e.g., CMOS based logic circuitry), firmware, software or any combination of hardware, firmware, and software (e.g., embodied in a machine readable medium). For example, the various electrical structure and methods may be embodied using transistors, logic gates, and electrical circuits (e.g., application specific integrated (ASIC) circuitry and/or in Digital Signal Processor (DSP) circuitry). In addition, it will be appreciated that the various operations, processes, and methods disclosed herein may be embodied in a machine-readable medium and/or a machine accessible medium compatible with a data processing system (e.g., a computer devices), and may be performed in any order (e.g., including using means for achieving the various operations). Accordingly, the specification and drawings are to be regarded in an illustrative manner rather than a restrictive sense.

What is claimed is:

1. A method of laser therapy comprising:
providing at least one substantially planar laser diode, configured to lase at a wavelength when driven, to form a medical instrument;
directing a resultant beam including an output beam of the at least one substantially planar laser diode at the corresponding wavelength thereof on a biological medium to impart energy to the biological medium; and
altering a mode of operation of the medical instrument when an interchangeable card of the medical instrument is replaced, the mode of operation comprising at least one segment including a time of pulsation of the at least one substantially planar laser diode and a frequency of pulsation of the at least one substantially planar laser diode.

2. The method of claim 1, wherein altering the mode of operation of the medical instrument when an interchangeable card of the medical instrument is removed further includes substituting the interchangeable card with a new interchangeable card.

3. The method of claim 1, further comprising providing a user control capability of the mode of operation of the medical instrument through an external connector configured to be able to connect to a computer device.

4. The method of claim 1, further comprising generating a soliton wave as the output beam of at least one substantially planar laser diode using a configuration of at least one substantially planar laser diode.

5. The method of claim 1, further comprising providing at least one light emitting diode (LED) to indicate an operational state of the medical instrument.

6. The method of claim 1, further comprising providing a display to indicate the mode of operation of the medical instrument.

7. The method of claim 1, further comprising at least one of:
controlling a power level of at least one substantially planar laser diode; and
sensing an operating current of at least one substantially planar laser diode.

8. The method of claim 1, further comprising audibly alerting at least one of an operational status of the medical instrument, a beginning of the mode of operation, a beginning of a segment, an end of the mode of operation, and an end of the segment.

9. The method of claim 1, further comprising selecting a stored mode of operation indicated through the display.

10. The method of claim 1, further comprising providing at least one infrared LED configured to emit infrared light during a duration of the mode of operation along with visible light from the at least one LED.

11. The method of claim 1, further comprising indicating a low battery state through at least one LED.

12. The method of claim 1, further comprising providing a capability to charge a battery of the medical instrument through the external connector.

13. The method of claim 1, further comprising sensing a temperature of at least one substantially planar laser diode.

14. The method of claim 1, further comprising sensing an ambient light external to the medical instrument to control a light intensity of at least one LED.

15. The method of claim 1, further comprising, prior to use of the medical instrument, at least one of:
programming the medical instrument with at least one mode of operation; and
calibrating at least one substantially planar laser diode.

16. The method of claim 1, further comprising providing an external memory to store a data indicating a diagnostic requirement of the medical instrument.

17. The method of claim 1, further comprising providing a capability to perform an external corrective diagnostic on the medical instrument through the external connector.

18. The method of claim 1, further comprising deactivating a faulty diode of the at least one substantially planar laser diode when the at least one substantially planar diode is a plurality of substantially planar laser diodes.

19. The method of claim 1, comprising providing an array of substantially planar laser diodes.

20. The method of claim 2, wherein substituting the interchangeable card with the new interchangeable card enables a mode of operation specific to a therapeutic condition associated with the new interchangeable card, and disables the mode of operation specific to a therapeutic condition associated with the interchangeable card.

21. The method of claim 3, wherein providing a user control capability of the mode of operation of the medical instrument through the external connector includes providing access to a network database including modes of operation through an encryption key.

22. The method of claim 21, further comprising:
selecting a mode of operation using the network database;
transmitting an encrypted information indicating the selected mode of operation to the medical instrument using the encryption key.

23. The method of claim 21, wherein providing access to the network database includes providing a user interface to the network database through an internet webpage.

24. The method of claim 22, further comprising decrypting the transmitted encrypted information to reprogram the medical instrument.

25. The method of claim 22, further comprising transferring a data indicating diagnostic information of the medical instrument to the network database.

26. The method of claim 23, further comprising sharing information indicating a mode of operation solely through the internet webpage between a medical instrument having a first encryption key and a medical instrument having a second encryption key,
wherein when the information indicating a mode of operation available through the internet webpage is encrypted and transmitted to the medical instrument having the first encryption key, access of the information indicating the mode of operation available through the internet webpage by the medical instrument having the second encryption key is prevented outside the internet webpage.

27. The method of claim 23, further comprising providing a feedback submission capability to a user of the medical instrument through the internet webpage.

28. The method of claim 25, wherein the data indicating diagnostic information of the medical instrument comprises at least one of:
a number of times the medical instrument is one of turned on and turned off;
a number of times the medical instrument is recharged;
a temperature variation of the medical instrument;
a number of times a mode of operation is used;
a data indicating a lifespan of the at least one substantially planar laser diode; and
a data from a real-time clock in the medical instrument indicating a history of use of the medical instrument.

29. A method of laser therapy comprising:
coupling a first medical instrument to a second medical instrument through a hardware connector, the first medical instrument and the second medical instrument each comprising at least one substantially planar laser diode configured to lase at a wavelength when driven;
altering a mode of operation of the first medical instrument and the second medical instrument when an interchangeable card of at least one of the first medical instrument and the second medical instrument is substituted with a new interchangeable card, the mode of operation comprising at least one segment including a time of pulsation of the at least one substantially planar laser diode and a frequency of pulsation of the at least one substantially planar laser diode;
enabling communication between the first medical instrument and the second medical instrument through the hardware connector; and
directing at least one of:
a first resultant beam including an output beam of the at least one substantially planar laser diode of the first medical instrument on a first location of a biological medium to impart energy to the first location of the biological medium; and
a second resultant beam including an output beam of the at least one substantially planar laser diode of the second medical instrument on a second location of the biological medium to impart energy to the second location of the biological medium.

30. The method of claim 29, comprising enabling communication between the first medical instrument and the second medical instrument through the hardware connector to at least one of:
providing compatibility to render the first medical instrument compatible with the second medical instrument; and
synchronizing the mode of operation between the first medical instrument and the second medical instrument.

31. The method of claim 29, further comprising generating a soliton wave as the output beam of both the at least one substantially planar laser diode of the first medical instrument and the second medical instrument.

32. The method of claim 29, further comprising providing an array of substantially planar laser diodes in each of the first medical instrument and the second medical instrument.

33. The method of claim 29, wherein enabling communication between the first medical instrument and the second medical instruments includes utilizing a communication protocol to facilitate the communication process.

34. The method of claim 30, wherein providing compatibility includes using the first medical instrument to power the second medical instrument.

35. The method of claim 30, wherein providing compatibility to render the first medical instrument compatible with the second medical instrument includes providing one of a step-up transformer circuit and a step-down transformer circuit to one of scale up and scale down a voltage level when the first medical instrument has an operating voltage different from that of the second medical instrument.

36. The method of claim 30, wherein synchronizing the mode of operation includes:
communicating, to the hardware connector, the mode of operation specific to a therapeutic condition associated with the new interchangeable card to be executed.

37. The method of claim 33, wherein utilizing a communication protocol to facilitate the communication process includes executing a cycle redundancy check to ensure integrity of the communication process.

38. The method of claim 33, wherein the hardware connector includes a flash programmable microcontroller comprising a flash memory to control the communication process.

39. The method of claim 34, wherein using the first medical instrument to power the second medical instrument includes turning off the at least one substantially planar laser diode of the first medical instrument prior to powering the second medical instrument.

40. The method of claim 36, further comprising:
preparing the first medical instrument and the second medical instrument to receive the mode of operation through the hardware connector; and
sending the mode of operation through the hardware connector to the first medical instrument and the second medical instrument bidirectionally at a same time.

41. The method of claim 40, further comprising executing a same set of instructions on both the first medical instrument and the second medical instrument.

42. The method of claim 40, further comprising at least one of:
time delaying the at least one segment of the mode of operation in one of the first medical instrument and the second medical instrument when compared to the at least one segment of the mode of operation in the other of the first medical instrument and the second medical instrument;
changing at least one of the time of pulsation and the frequency of pulsation of the at least one substantially planar laser diode of one of the first medical instrument and the second medical instrument when compared to the other of the first medical instrument and the second medical instrument; and
executing a different at least one segment on the first medical instrument when compared to the at least one segment executed concurrently on the second medical instrument.

43. The method of claim 40, further comprising using one of the first medical instrument and the second medical instrument to indicate an end of the mode of operation to the hardware connector.

44. A medical instrument comprising:
at least one substantially planar laser diode, configured to lase at a wavelength when driven; and
an interchangeable card to alter a mode of operation of the medical instrument when removed, the mode of operation comprising at least one segment including a time of pulsation of the at least one substantially planar laser diode, and a frequency of pulsation of the at least one substantially planar laser diode,
wherein a resultant beam including an output beam of the at least one substantially planar laser diode at the corresponding wavelength thereof is focused on a biological medium to impart energy to the biological medium.

45. The medical instrument of claim 44, further comprising an external connector configured to be able to connect to a computer device to provide a user control capability of the mode of operation of the medical instrument.

46. The medical instrument of claim 44, wherein the interchangeable card is substituted with a new interchangeable card.

47. The medical instrument of claim 44, comprising an array of substantially planar laser diodes.

48. The medical instrument of claim 44, wherein the device is configured to generate a soliton wave as the output beam of the at least one substantially planar laser diode.

49. The medical instrument of claim 44, further comprising at least one LED to indicate an operational state of the medical instrument.

50. The medical instrument of claim 44, further comprising a display to indicate the mode of operation of the medical instrument.

51. The medical instrument of claim 44, further comprising a rechargeable battery to power the medical instrument.

52. The medical instrument of claim 44, further comprising a button to one of turn on and turn off the medical instrument and to select the mode of operation.

53. The medical instrument of claim 44, further comprising an audio speaker to alert at least one of an operational status of the medical instrument, a beginning of the mode of operation, a beginning of a segment, an end of the mode of operation, and an end of the segment.

54. The medical instrument of claim 44, further comprising an external memory to store a data indicating a diagnostic requirement of the medical instrument.

55. The medical instrument of claim 44, further comprising a real-time clock to track a history of use of the medical instrument.

56. The medical instrument of claim 44, further comprising at least one infrared LED configured to emit infrared light during a duration of the mode of operation along with visible light from the at least one LED.

57. The medical instrument of claim 44, wherein the medical instrument is a probe device.

58. The medical instrument of claim 44, further comprising a housing appropriately shaped and sized for convenient portability of the medical instrument.

59. The medical instrument of claim 44, wherein the medical instrument, prior to a use thereof, is programmed with at least one mode of operation, and the at least one substantially planar laser diode is calibrated.

60. The medical instrument of claim 44, wherein an output power of the at least one substantially planar laser diode is one of ˜5 mW, ˜50 mW, and ˜500 mW.

61. The medical instrument of claim 44, wherein the lasing wavelength of the at least one substantially planar laser diode is one of ˜650 nm, ˜660 nm, ˜780 nm, and ˜808 nm.

62. The medical instrument of claim 45, wherein a user access to a network database including modes of operation is provided through the external connector using an encryption key.

63. The medical instrument of claim 46, wherein a mode of operation specific to a therapeutic condition associated with the new interchangeable card is enabled, and the mode of operation specific to a therapeutic condition associated with the interchangeable card is disabled, when the interchangeable card is substituted with the new interchangeable card.

64. The medical instrument of claim 49, further comprising a controller to at least one of:
control a power level of the at least one substantially planar laser diode;
sense an operating current of the at least one substantially planar laser diode;
monitor a measurement of a light sensor of an ambient light external to the medical instrument to control a light intensity of visible light from the at least one LED;
control a laser driver to the at least one substantially planar laser diode;
control a power management circuitry of the at least one substantially planar laser diode; and
control a temperature of the at least one substantially planar laser diode.

65. The medical instrument of claim 51, wherein the rechargeable battery is a Lithium-Ion battery.

66. The medical instrument of claim 51, further comprising a battery charger including a safety circuitry to monitor a charge current and a maximum allowed voltage while charging the rechargeable battery.

67. The medical instrument of claim 51, wherein a low battery state is indicated through the at least one LED.

68. The medical instrument of claim 51, wherein the external connector is a multi-use connector that, in addition to being configured to be able to connect to a computer device, is used to at least one of:
program the medical instrument;
calibrate the at least one substantially planar laser diode;
perform a corrective diagnostic of the medical instrument;
couple the medical instrument to another medical instrument; and
charge the rechargeable battery of the medical instrument.

69. The medical instrument of claim 52, further comprising a reset controller to control a resetting of the medical instrument, the resetting being accomplished through pressing the button for a time period exceeding a threshold time period.

70. The medical instrument of claim 54, wherein the data indicating the diagnostic requirement of the medical instrument comprises at least one of:
a number of times the medical instrument is one of turned on and turned off;
a number of times the medical instrument is recharged;
a temperature variation of the medical instrument;
a number of times a mode of operation is used;
a data indicating a lifespan of the at least one substantially planar laser diode; and
a data indicating a history of use of the medical instrument.

71. The medical instrument of claim 57, wherein the probe device comprises a storage card to store modes of operation of the probe device.

72. The medical instrument of claim 58, wherein the at least one substantially planar laser diode is mounted on a recessed portion of the housing of the medical instrument.

73. The medical instrument of claim 59, wherein the medical instrument is reprogrammed with an encrypted mode of operation obtained from the network database.

74. A therapeutic laser system comprising:
a first medical instrument;
a second medical instrument,
wherein the first medical instrument and the second medical instrument each comprise at least one substantially planar laser diode configured to lase at a wavelength when driven;
a hardware connector to couple the first medical instrument to the second medical instrument, and to enable communication between the first medical instrument and the second medical instrument; and
an interchangeable card of at least one of the first medical instrument and the second medical instrument to alter a mode of operation of the first medical instrument and the second medical instrument when the interchangeable card is substituted with a new interchangeable card, the mode of operation comprising at least one segment including a time of pulsation of the at least one substantially planar laser diode and a frequency of pulsation of the at least one substantially planar laser diode,
wherein at least one of:
a first resultant beam including an output beam of the at least one substantially planar laser diode of the first medical instrument; and
a second resultant beam including an output beam of the at least one substantially planar laser diode of the second medical instrument is focused on one of a first location of a biological medium and a second location of the biological medium to impart energy to one of the first location of the biological medium and the second location of the biological medium.

75. The therapeutic laser system of claim 74, wherein a mode of operation specific to a therapeutic condition associated with the new interchangeable card is enabled, and the mode of operation specific to a therapeutic condition associated with the interchangeable card is disabled, when the interchangeable card is substituted with the new interchangeable card.

76. The therapeutic laser system of claim 74, wherein communication between the first medical instrument and the second medical instrument is enabled through the hardware connector to at least one of:
render the first medical instrument compatible with the second medical instrument; and
synchronize the mode of operation between the first medical instrument and the second medical instrument.

77. The therapeutic laser system of claim 74, wherein a soliton wave is generated as the output beam of both the at least one substantially planar laser diode of the first medical instrument and the second medical instrument.

78. The therapeutic laser system of claim 74, wherein the first medical instrument is used to power the second medical instrument.

79. The therapeutic laser system of claim 74, wherein the first medical instrument and the second medical instrument each comprise an array of substantially planar laser diodes.

80. The therapeutic laser system of claim 74, wherein one of the first medical instrument and the second medical instrument is a probe device.

81. The therapeutic laser system of claim 74, where a communication protocol is utilized to facilitate the communication process between the first medical instrument and the second medical instrument.

82. The therapeutic laser system of claim 74, wherein one of a step-up transformer circuit and a step-down transformer circuit is provided to one of scale up and scale down a voltage level when the first medical instrument has an operating voltage different from that of the second medical instrument.

83. The therapeutic laser system of claim 74, wherein an output power of the at least one substantially planar laser diode is one of ˜5 mW, ˜50 mW, and ˜500 mW.

84. The therapeutic laser system of claim 78, wherein the at least one substantially planar laser diode of the first medical instrument is turned off prior to powering the second medical instrument.

85. The therapeutic laser system of claim 81, wherein a cycle redundancy check is executed to ensure integrity of the communication process.

86. The therapeutic laser system of claim 81, wherein the hardware connector includes a flash programmable microcontroller comprising a flash memory to control the communication process.

87. The therapeutic laser system of claim 81, wherein:
the mode of operation to be executed in the first medical instrument and the second medical instrument is communicated to the hardware connector,
the first medical instrument and the second medical instrument are prepared to receive the mode of operation through the hardware connector; and
the mode of operation is sent through the hardware connector to the first medical instrument and the second medical instrument bidirectionally at a same time,
during synchronization of the mode of operation.

88. The therapeutic laser system of claim 87, wherein a same set of instructions is executed on both the first medical instrument and the second medical instrument.

89. The therapeutic laser system of claim 87, wherein:
at least one segment of the mode of operation in one of the first medical instrument and the second medical instrument is time delayed compared to the at least one segment of the mode of operation in the other of the first medical instrument and the second medical instrument;
at least one of the time of pulsation and the frequency of pulsation of the at least one substantially planar laser diode of one of the first medical instrument and the second medical instrument is changed when compared to the other of the first medical instrument and the second medical instrument; and
a different at least one segment is executed on the first medical instrument compared to the at least one segment executed concurrently on the second medical instrument,
during synchronization of the mode of operation.

90. The therapeutic laser system of claim 87, wherein one of the first medical instrument and the second medical instrument is used to indicate an end of the mode of operation to the hardware connector.
